(19) United States
(12) Patent Application Publication
(10) Pub. No.: US 2024/0279701 A1
(43) Pub. Date: Aug. 22, 2024

(54) Preparation Method and Enrichment Method for Cyanidin-3-Diglucoside-5-Glucoside

(71) Applicants: INSTITUTE OF QUALITY STANDARD AND TESTING TECHNOLOGY FOR AGRO-PRODUCTS OF CHINESE ACADEMY OF AGRICUL, Beijing (CN); China Agricultural University, Beijing (CN)

(72) Inventors: Zhenzhen XU, Beijing (CN); Shini YANG, Beijing (CN); Xiaojun LIAO, Beijing (CN)

(21) Appl. No.: 18/393,145

(22) Filed: Dec. 21, 2023

(30) Foreign Application Priority Data
Feb. 17, 2023 (CN) 2023101531986

(51) Int. Cl. C12P 19/44

(52) U.S. Cl. CPC C12P 19/44

(57) ABSTRACT

The present disclosure discloses a preparation method and enrichment method of cyanidin-3-diglucoside-5-glucoside, which relates to the technical field of biochemical industry. Enzyme is used to catalyze the substrate to obtain cyanidin-3-diglucoside-5-glucoside. The temperature of the catalytic reaction is 20-40° C., and the reaction time is 10-30 h. This enzyme can convert various anthocyanins into cyanidin-3-diglucoside-5-glucoside, thereby increasing the content of target anthocyanin. The method is simple to operate, specifically improves the content of cyanidin-3-diglucoside-5-glucoside in the crude extract, and reduces the types of anthocyanins in the crude extract to a certain extent, thereby reducing difficulty of subsequent separation and purification. The use of organic reagents and equipment in the traditional enrichment and purification process is reduced, which is energy-saving and environmentally friendly, and the product obtained by catalysis is of good quality, high yield and high purity.

### TECHNICAL FIELD

The present disclosure relates to the field of biochemical engineering technology, in particular to a preparation method and an enrichment method for cyanidin-3-diglucoside-5-glucoside.

### SEQUENCE LISTING

The instant application contains a Sequence Listing which has been submitted electronically in XML format and is hereby incorporated by reference in its entirety. Said XML copy, created on Dec. 21, 2023, is named 51649-004001_Sequence_Listing_12_21_23.xml and is 4,059 bytes in size.

### BACKGROUND ART

Anthocyanin is water-soluble pigment that widely exists in plants. In addition to bringing various colors to plants and fruits, anthocyanin has various biological activities such as anti-oxidation, anti-fatigue, anti-aging, anti-tumor, prevention of cardiovascular and cerebrovascular diseases and so on, which has great development prospects in food, cosmetics, medicine and other fields.

At present, the production of anthocyanin mainly relies on the traditional solvent extraction method. Due to the limitations in raw material prices and extraction efficiency, anthocyanin extract and its high-purity monomer are expensive. There are more than 600 kinds of anthocyanins isolated and identified from nature, but the commercialized anthocyanin monomers are extremely rare. There are only more than 30 kinds of commercialized products, the number of which is about only 5% of the total. The companies that produce anthocyanin monomers worldwide are less than 10, such as “Polyphenols Laboratories” in Norway, “INDOFINE Chemical” and “ChromaDex” in the United States, “Extrasynthese” in France and “Shanghai Tongtian” in China, etc. Insufficient types of commercialized anthocyanin monomers have greatly restricted the research related to the physiological functions and action mechanisms of anthocyanins, and have limited their market application.

At present, the methods of extracting and purifying anthocyanins from plants have made great progress. The innovative extraction techniques, including ultrasound, microwave, pulsed electric field, and high pressure carbon dioxide have been applied to improve the total extraction rate of anthocyanins. However, none of the methods for extracting anthocyanins from plant substrates has specific selectivity for anthocyanins, and due to the similarity of properties between anthocyanin monomers, the extracted anthocyanins are usually a mixture containing various molecules and different chemical structures. Therefore, it is an urgent problem to be solved in the anthocyanin production industry to further develop low-cost and high-quality raw materials for extraction, and have breakthrough in environment-friendly separation and extraction technology and high-purity monomer preparation technology.

The existing patent application entitled “3,5-Disaccharide Anthocyanin Preparation Method” uses a method of combining gel columns and macroporous resins to separate anthocyanin monomers, which uses large amount of organic solvents, resulting in serious waste of resources and cumbersome operation, which is not suitable for large-scale industrial production. The patent application entitled “Method for Extracting Anthocyanins from Black Rice Bran Using Microwave-Assisted Method” adopts microwave-assisted technology to increase the total amount of anthocyanins extracted, but it cannot specifically increase the content of a specific anthocyanin, and the subsequent separation and purification processes remain complex.

In view of this, the present disclosure is specifically proposed.

### SUMMARY

The purpose of the present disclosure is to provide a preparation method and enrichment method of cyanidin-3-diglucoside-5-glucoside to solve the above technical problems, thereby greatly reducing the difficulty of subsequent preparation and purification.

The present disclosure is achieved as follows.

In the first aspect, the present disclosure provides a method for preparing cyanidin-3-diglucoside-5-glucoside, which includes the following steps: performing catalytic reaction for the substrate by using enzyme, to obtain cyanidin-3-diglucoside-5-glucoside, wherein the temperature of the catalytic reaction is 20-40° C., and the duration of the catalytic reaction is 10-30 h.

The enzyme is produced by biosynthesis. The preparation method of the enzyme includes introducing the carrier of the target gene containing the nucleotide sequence as set forth in SEQ ID NO. 1 or having at least more than 90%, 91%, 92%, 93%, 94%, 95%, 96%, 97%, 98% or 99% sequence identity to SEQ ID NO. 1 and having activity of hydrolysis of acylated anthocyanin into engineering bacteria, and then selecting positive single colonies for cultivation, induction, and lysis to obtain enzyme.

The substrate is anthocyanin. The nucleotide sequence of the enzyme is set forth in SEQ ID NO. 1 with UniProt ID of Q53547 or has at least more than 90%, 91%, 92%, 93%, 94%, 95%, 96%, 97%, 98% or 99% sequence identity to SEQ ID NO. 1 and has activity of hydrolysis of acylated anthocyanin.

For the first time, the catalysis of an enzyme is used by inventor to target enrich a certain anthocyanin. This enzyme can convert a variety of anthocyanins into cyanidin-3-diglucoside-5-glucoside, thereby improving the content of target anthocyanin. The method is easy to operate, specifically increases the content of cyanidin-3-diglucoside-5-glucoside in the crude extract, and reduces the types of anthocyanins in the crude extract (such as plant extraction solution) in a certain extent, thereby reducing the difficulty of subsequent separation and purification. Therefore, the use of organic reagents and equipment in the traditional enrichment and purification process is reduced, which is energy-saving and environmentally friendly, and the product obtained by catalysis is of good quality, high yield and high purity.

The above catalytic reaction is performed in a mild condition, such that the targeted enrichment and preparation of cyanidin-3-diglucoside-5-glucoside can be realized in conventional laboratories and factories.

In a preferred embodiment of the present disclosure, the above-mentioned anthocyanin is at least one selected from glycosylated anthocyanins, acylated anthocyanins, aggregates of small molecular weight of anthocyanin, mixture of anthocyanins or crude extract of anthocyanins. The concentration of anthocyanins in the catalytic reaction system is 1-100 mg/mL.

In a preferred embodiment of the present disclosure, the above-mentioned anthocyanin is any one selected from cyanidin-3-coumaroyl-diglucoside-5-O-glucoside or the following anthocyanin extracts, including honeyberry extract, blueberry fruit extract, radish extract, purple cabbage extract and purple sweet potato extract.

In a preferred embodiment of the present disclosure, when the substrate is cyanidin-3-coumaroyl-diglucoside-5-O-glucoside, the concentration (the final concentration of substrate actually participating in the reaction) of the substrate in the reaction system is 1-100 mg/mL, and the concentration of enzyme is 0.5-2 mg/mL. Under the above condition, the catalytic efficiency is higher, and the yield of the product obtained by catalysis is higher.

In a preferred embodiment of the present disclosure, when the substrate is a honeyberry extract, a blueberry fruit extract, a radish extract, a purple cabbage extract or a purple sweet potato extract, the concentration (the final concentration of the substrate actually participating in the reaction) of the substrate in the reaction system is 10-500 mg/mL, and the concentration of the enzyme is 0.5-2 mg/mL.

Preferably, the pH of the reaction system is 4.0-8.0. Under the above condition, the catalytic efficiency is higher, and the yield of the product obtained by catalysis is higher. For example, the pH is adjusted to 6-8.

In a preferred embodiment of the present disclosure, the extract is extracted by citric acid aqueous solution or acidified ethanol.

In a preferred embodiment of the present disclosure, the preparation method of purple cabbage extracts via citric acid aqueous solution is as follows: mixing crushed purple cabbage with citric acid aqueous solution at the ratio of material to liquid from 1:2 to 1:10, and performing immersing and extracting at 50-60° C. for 20-30 min.

Under the above extraction condition, the total anthocyanin content in the extraction solution has a relatively high level.

In a preferred embodiment of the present disclosure, the engineering bacteria are selected from Escherichia coli (E. coli).

The term “engineering bacteria” refers to any bacteria that can be transformed or transfected with exogenous nucleic acid, such as prokaryotes (e.g., E. coli).

In the second aspect, the present disclosure provides use of biological enzyme in the enrichment of cyanidin-3-diglucoside-5-glucoside. The preparation method of biological enzyme includes introducing the carrier containing the target gene of the nucleotide sequence as set forth in SEQ ID NO. 1 or having at least more than 90%, 91%, 92%, 93%, 94%, 95%, 96%, 97%, 98% or 99% sequence identity to SEQ ID NO. 1 and having activity of hydrolysis of acylated anthocyanin into the engineering bacteria, and then selecting the positive single colony for cultivation, induction, and lysis to obtain the enzyme.

Use includes using enzyme to catalyze the substrate to obtain cyanidin-3-diglucoside-5-glucoside, wherein the temperature of the catalytic reaction is 20-40° C., the duration of the catalytic reaction is 10-30 h, and the substance is selected from anthocyanins.

The temperature of the catalytic reaction is 20° C., 25° C., 30° C., 35° C. or 40° C. The duration of the catalytic reaction is 10 h, 12 h, 15 h, 20 h, 25 h or 30 h.

The methods in the above steps of cultivation, induction, and lysis to obtain the enzyme are all conventional technical means in the art, and those skilled in the art can make selections following the usual practice.

In the third aspect, the present disclosure provides a kit for enriching cyanidin-3-diglucoside-5-glucoside, which includes a biological enzyme, wherein the biological enzyme is prepared by the following method: introducing the carrier containing the target gene of the nucleotide sequence as set forth in SEQ ID NO. 1 or having at least more than 90%, 91%, 92%, 93%, 94%, 95%, 96%, 97%, 98% or 99% sequence identity to SEQ ID NO. 1 and having activity of hydrolysis of acylated anthocyanin into the engineering bacteria, and then selecting the positive single colony for cultivation, induction, and lysis to obtain the enzyme.

In other embodiments, the biological enzyme in the above kit is in the form of freeze-dried powder, liquid reagent and the like.

The present disclosure has the following beneficial effects.

The present disclosure utilizes the catalysis of an enzyme to carry out targeted enrichment of a certain anthocyanin for the first time, wherein the enzyme can convert various anthocyanins into cyanidin-3-diglucoside-5-glucoside, thereby improving the content of target anthocyanin. The method is simple to operate, and specifically increases the content of cyanidin-3-diglucoside-5-glucoside in the crude extract, and reduces the types of anthocyanins in the crude extract (such as plant extraction solution) to a certain extent, thus reducing the difficulty of subsequent separation and purification. The use of organic reagents and equipment in the traditional enrichment and purification process is reduced, which is energy-saving and environmentally friendly, the product obtained by catalysis is of good quality, high yield and high purity.

### BRIEF DESCRIPTION OF DRAWINGS

In order to illustrate the technical solutions of the embodiments of the present disclosure more clearly, the drawings used in the embodiments will be briefly introduced below. It should be understood that the following drawings only show some embodiments of the present disclosure, and therefore, should not be regarded as a limitation on the scope, and those ordinarily skilled in the art can also obtain other related drawings based on these drawings without inventive effort.

FIGS. 1A and 1B show a high performance liquid chromatography of high-purity cyanidin-3-diglucoside-5-glucoside prepared with cyanidin-3-coumaroyl-diglucoside-5-O-glucoside as substrate;

FIGS. 2A and 2B show a high performance liquid chromatography of cyanidin-3-diglucoside-5-glucoside prepared with purple cabbage leaf extract as substrate;

FIGS. 3A and 3B show a high performance liquid chromatography of cyanidin-3-diglucoside-5-glucoside prepared with purple cabbage leaf extract as substrate, wherein the pH of catalytic reaction is 3.0; and

FIGS. 4A and 4B show a high performance liquid chromatography of cyanidin-3-diglucoside-5-glucoside prepared with purple cabbage leaf extract as substrate, wherein the duration of the catalytic reaction is 8 h.

### DETAILED DESCRIPTION OF EMBODIMENTS

In order to make the purpose, technical solutions and advantages of the embodiments of the present disclosure clearer, the technical solutions in the embodiments of the present disclosure will be clearly and completely described below. Those without specific conditions indicated in the examples are carried out according to the conventional conditions or the conditions suggested by the manufacturer. The reagents or instruments used without manufacturer indicated are all conventional products that could be purchased from the market.

The characteristics and performance of the present disclosure will be described in further detail below in conjunction with the examples.

### Example 1

The example provided a preparation method for cyanidin-3-diglucoside-5-glucoside.

1) Preparation for enzyme: a codon optimization for the nucleotide sequence (SEQ ID NO. 1), which was reported to be able to convert a variety of acylated anthocyanins into cyanidin-3-diglucoside-5-glucoside, was performed and the gene was synthesized. The synthetic gene sequence was cloned into the Pcold-TF vector and then transformed in the Escherichia coli BL21 (DE3). The transformed cell cultures were inoculated into LB medium, and the IPTG was added to induce the expression of the enzyme when the OD₆₀₀ of the culture was between 0.6 and 0.8, which was incubated overnight. The bacteria were collected by centrifugation, which was resuspended, and then crushed by ultrasonic, the supernatant was collected by centrifugation, and the target enzyme solution was obtained after purification.

2) The cyanidin-3-coumaroyl-diglucoside-5-O-glucoside was used as the substrate. The concentration of the substrate in each 200 μL of substrate system was 10 mg/mL. The concentration of the enzyme prepared in step 1) was 1 mg/mL. At room temperature, the pH was adjusted to 7.0, and the reaction was performed for 12 h. The reaction product was collected and detected by HPLC.

The separated and purified product was identified by high performance liquid chromatography (HPLC) for qualitative and quantitative detection. The specific chromatographic parameters were as follows: mobile phase A was 5% formic acid aqueous solution, mobile phase B was acetonitrile, chromatographic column was Poroshell 120 SB-C18 (3*100 mm, 2.7 mm), column temperature was 40° C., and flow rate was 0.4 mL/min. The UV detector had a detection wavelength of 520 nm.

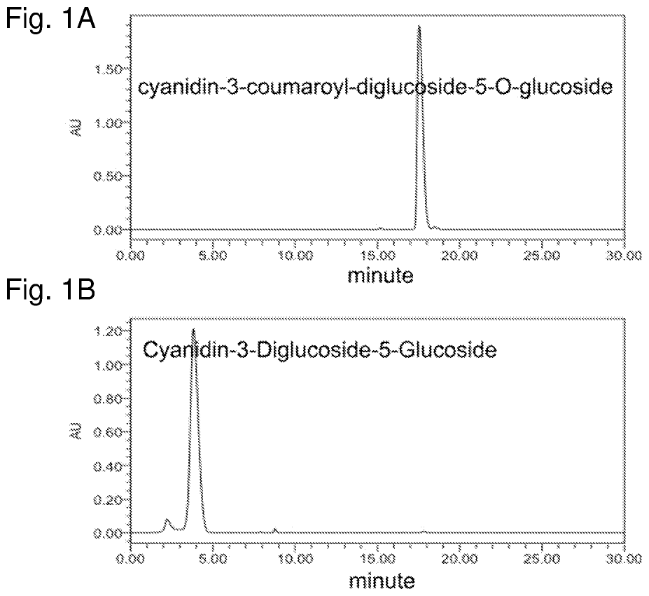

The results in FIG. 1 show that 95% of the substrate (cyanidin-3-coumaroyl-diglucoside-5-O-glucoside) can be converted to generate cyanidin-3-diglucoside-5-glucoside. The conversion rate is relatively high.

### Example 2

The example provided an enrichment method for cyanidin-3-diglucoside-5-glucoside.

Fresh purple cabbage was cut into small pieces, and crushed in a high-speed wall-breaking machine for 1 min. The citric acid-water, as an extraction solvent, at a ratio of 1:5 was added into the crushed purple cabbage leaves, and the resultant was immersed and extracted at 60° C. for 30 min. The resultant was filtered through 200-mesh gauze to remove the residue and obtain anthocyanin crude extraction solution. Purple cabbage anthocyanin dry powder was obtained after spray drying. The anthocyanin crude extract with a total anthocyanin content of 1.5 g/100 g was taken as the substrate. In a 200 μL reaction system, the final concentration of the substrate was 60 mg/ml. 1 mg/ml of enzyme solution prepared through step (1) of Example 1 was added, the pH of the resultant was adjusted to 6.5 at room temperature, the reaction lasted for 12 h, and the supernatant was obtained by centrifugation, which was the enriched cyanidin-3-diglucoside-5-glucoside solution. The reaction product was detected by HPLC.

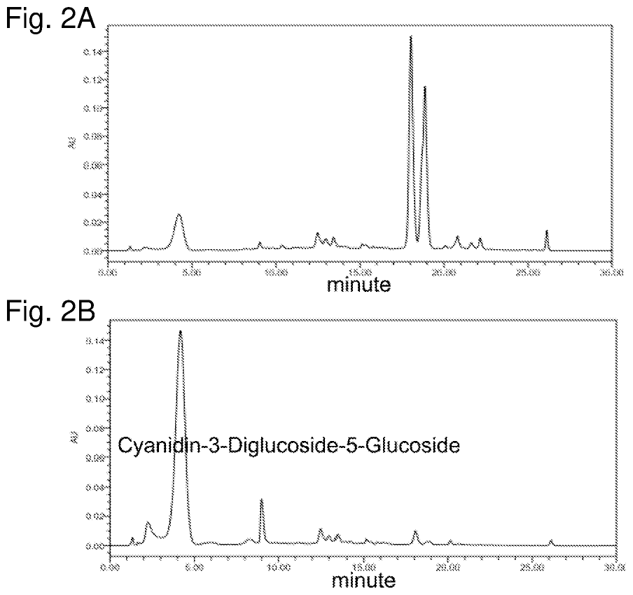

The result is shown in FIG. 2, which shows that the content of cyanidin-3-diglucoside-5-O-glucoside of product was increased from 0.05 mg/ml to 0.67 mg/mL, and the proportion was increased from 6.1% to 78.9% (FIG. 2).

### Example 3

Preparation for enzyme (SEQ ID NO. 2) which have 99% similar amino sequences with SEQ. 1.

Fresh purple cabbage was cut into small pieces, and crushed in a high-speed wall-breaking machine for 1 min. The citric acid-water, as an extraction solvent, at a ratio of 1:5 was added into the crushed purple cabbage leaves, and the resultant was immersed and extracted at 60° C. for 30 min. The resultant was filtered through 200-mesh gauze to remove the residue and obtain anthocyanin crude extraction solution. Purple cabbage anthocyanin dry powder was obtained after spray drying. The anthocyanin crude extract with a total anthocyanin content of 1.5 g/100 g was taken as the substrate. In a 200 μL reaction system, the final concentration of the substrate was 60 mg/ml. 1 mg/mL of enzyme solution prepared through step (1) of Example 1 was added, the pH of the resultant was adjusted to 6.5 at room temperature, the reaction lasted for 12 h, and the supernatant was obtained by centrifugation, which was the enriched cyanidin-3-diglucoside-5-glucoside solution. The reaction product was detected by HPLC. The result shows that the content of cyanidin-3-diglucoside-5-O-glucoside of product was increased from 0.05 mg/mL to 0.67 mg/mL, and the proportion was increased from 6.1% to 74.7%

### Example 4

The example provided an enrichment method for cyanidin-3-diglucoside-5-glucoside.

Fresh purple cabbage was cut into small pieces, and crushed in a high-speed wall-breaking machine for 1 min. The citric acid-water, as an extraction solvent, at a ratio of 1:10 was added to the crushed purple cabbage leaves, and the resultant was immersed and extracted at 60° C. for 30 min. The resultant was filtered through 200-mesh gauze to remove residue and obtain anthocyanin crude extraction solution. Purple cabbage anthocyanin dry powder was obtained after spray drying. The anthocyanin crude extract with a total anthocyanin content of 1.5 g/100 g was taken as the substrate. In the 200 μL of substrate system, the final concentration of the substrate was 60 mg/mL, and 1 mg/mL enzyme solution prepared through step (1) of Example 1 was added. The pH of the resultant was adjusted to 6.0 at room temperature, the reaction lasted for 12 h, and the supernatant was obtained by centrifugation, which was the enriched cyanidin-3-diglucoside-5-glucoside solution. The reaction product was detected by HPLC. The results show that the content of cyanidin-3-diglucoside-5-O-glucoside of the product was increased from 0.05 mg/mL to 0.59 mg/mL, and the proportion was increased from 6.1% to 69.4%.

### Example 5

The example provided an enrichment method for cyanidin-3-diglucoside-5-glucoside.

Fresh purple cabbage was cut into small pieces, and crushed in a high-speed wall-breaking machine for 1 min. The citric acid-water, as an extraction solvent, at a ratio of 1:10 was added to the crushed purple cabbage leaves, and the resultant was immersed and extracted at 60° C. for 30 min. The resultant was filtered through 200-mesh gauze to remove residue and obtain anthocyanin crude extraction solution. Purple cabbage anthocyanin dry powder was obtained after spray drying. The anthocyanin crude extract with a total anthocyanin content of 1.5 g/100 g was taken as the substrate. In the 200 μL of substrate system, the final concentration of the substrate was 60 mg/mL, and 2 mg/mL enzyme solution prepared through step (1) of Example 1 was added. The pH of the resultant was adjusted to 6.5 at room temperature, the reaction lasted for 15 h, and the supernatant was obtained by centrifugation, which was the enriched cyanidin-3-diglucoside-5-glucoside solution. The reaction product was detected by HPLC. The results show that the content of cyanidin-3-diglucoside-5-glucoside of the product was increased from 0.05 mg/ml to 0.65 mg/mL, and the proportion was increased from 6.1% to 76.4%.

### Comparative Example 1

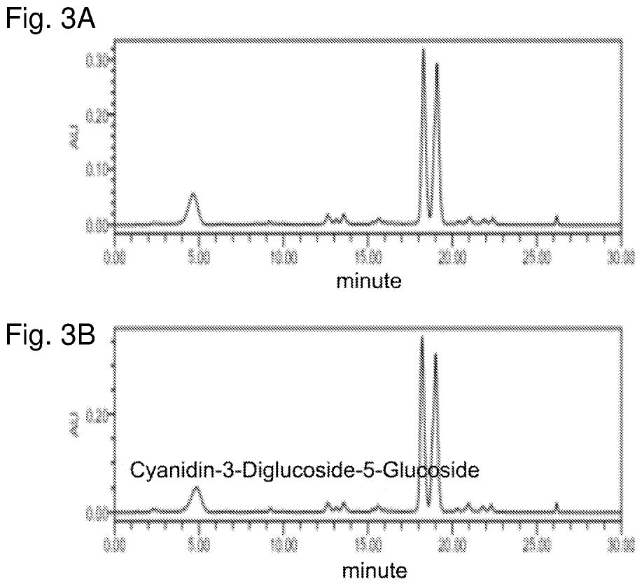

Compared with Example 2, the difference only was that the pH of the catalytic reaction was different, and the rest of the conditions were the same. The pH of catalytic reaction of the comparative example was 3.0. The results show that the substrate was not converted, and the cyanidin-3-diglucoside-5-glucoside content of product was not increased (referring to FIG. 3).

### Comparative Example 2

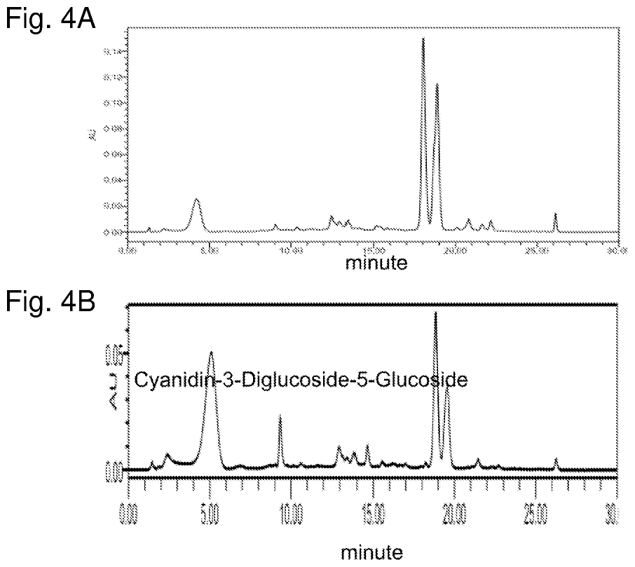

Compared with Example 2, the difference only was that the duration of the catalytic reaction was different, and the rest of the conditions were the same. The duration of catalytic reaction of the comparative example was 8 h. The cyanidin-3-diglucoside-5-glucoside content of the product was increased from 0.05 mg/mL to 0.32 mg/mL, and the proportion was increased from 6.5% to 37.6% (referring to FIG. 4).

To sum up, under the conditions provided by the present disclosure, the catalytic efficiency is relatively high, and the yield of the product obtained by catalysis is relatively high. The above descriptions are only preferred embodiments of the present disclosure, and are not intended to limit the present disclosure. For those skilled in the art, the present disclosure can have various modifications and changes. Any modifications, equivalent replacements, improvements, etc. made within the spirit and principles of the present disclosure shall be included within the protection scope of the present disclosure.

1. A preparation method for cyanidin-3-diglucoside-5-glucoside, wherein the preparation method comprises following steps: performing a catalytic reaction for a substrate by using enzyme, to obtain the cyanidin-3-diglucoside-5-glucoside, wherein a temperature of the catalytic reaction is 20-40° C., and a duration of the catalytic reaction is 10-30 h;
the enzyme is produced by biosynthesis, wherein a preparation method for the enzyme comprises introducing a carrier of a target gene containing a nucleotide sequence as set forth in SEQ ID NO. 1 or having at least more than 90%, 91%, 92%, 93%, 94%, 95%, 96%, 97%, 98% or 99% sequence identity to SEQ ID NO. 1 and having activity of hydrolysis of acylated anthocyanin into an engineering bacterium, and then selecting positive single colonies for cultivation, induction, and lysis to obtain the enzyme; and
the substrate is selected from anthocyanins.

2. The preparation method according to claim 1, wherein the anthocyanin is at least one selected from glycosylated anthocyanins, acylated anthocyanins, aggregates of small molecular weight of anthocyanins, a mixture of anthocyanins or a crude extract of anthocyanins, and a concentration of the anthocyanin in a system of the catalytic reaction is 1-100 mg/mL.

3. The preparation method according to claim 2, wherein the anthocyanin is any one selected from cyanidin-3-coumaroyl-diglucoside-5-O-glucoside or following anthocyanin extracts, comprising a honeyberry extract, a blueberry fruit extract, a radish extract, a purple cabbage extract and a purple sweet potato extract.

4. The preparation method according to claim 3, wherein when the substrate is the cyanidin-3-coumaroyl-diglucoside-5-O-glucoside, a concentration of the substrate in a reaction system is 1-100 mg/mL, and a concentration of the enzyme is 0.5-2 mg/mL.

5. The preparation method according to claim 3, wherein when the substrate is selected from the honeyberry extract, the blueberry fruit extract, the radish extract, the purple cabbage extract or the purple sweet potato extract, a concentration of the substrate in a reaction system is 100-500 mg/mL, and a concentration of the enzyme is 0.5-2 mg/mL.

6. The preparation method according to claim 5, wherein the extract is extracted by a citric acid aqueous solution or acidified ethanol.

7. The preparation method according to claim 6, wherein a preparation method for the purple cabbage extract via the citric acid aqueous solution is as follows: mixing crushed purple cabbage or purple sweet potato with the citric acid aqueous solution at a ratio of material to liquid from 1:2 to 1:10, and performing immersing and extracting at 50-60° C. for 20-30 min.

8. The preparation method according to claim 1, wherein the engineering bacterium is selected from Escherichia coli.

9. Use of a biological enzyme in enriching cyanidin-3-diglucoside-5-glucoside, wherein a preparation method for the biological enzyme comprises introducing a carrier containing a target gene of a nucleotide sequence as set forth in SEQ ID NO. 1 or having at least more than 90%, 91%, 92%, 93%, 94%, 95%, 96%, 97%, 98% or 99% sequence identity to SEQ ID NO. 1 and having activity of hydrolysis of acylated anthocyanin into an engineering bacterium, and then selecting positive single colonies for cultivation, induction, and lysis to obtain the enzyme, wherein
the use comprises using the enzyme to perform a catalytic reaction on a substrate to obtain the cyanidin-3-diglucoside-5-glucoside, wherein a temperature of the catalytic reaction is 20-40° C., and a duration of the catalytic reaction is 10-30 h; and
the substrate is selected from anthocyanins.

10. A kit for enriching cyanidin-3-diglucoside-5-glucoside, wherein the kit comprises a biological enzyme, wherein the biological enzyme is prepared by a following method: introducing a carrier of a target gene containing a nucleotide sequence as set forth in SEQ ID NO. 1 or having at least more than 90%, 91%, 92%, 93%, 94%, 95%, 96%, 97%, 98% or 99% sequence identity to SEQ ID NO. 1 and having activity of hydrolysis of acylated anthocyanin into an engineering bacterium, and then selecting positive single colonies for cultivation, induction, and lysis to obtain the enzyme.

11. The preparation method according to claim 5, wherein a pH of the reaction system is 4.0-8.0.


SEQUENCE LISTING (2 sequences)

SEQ ID NO: 1 - DNA, 657 bp, synthetic construct
  source 1..657: mol_type = other DNA
atgaccgaacccttgattcttcagcccgccaagccggcagacgcctgcgtaatctggttgcatggcctgggtgccgatcgctacgactttatgccggtagccgaagccctgcaggaaagcctgctgaccacgcgcttcgtcttgccccaggcgccgacacgcccggtgaccatcaacggcggctacgagatgccgagctggtacgacatcaaggccatgagcccggcccgttcgatcagcctggaggagctggaagtgtcggccaaaatggtcacggatctgatcgaagcgcagaagagaaccggaatagacgcctctcggattttcctcgccggtttttcccagggtggcgccgtggttttccacacggcgtttataaattggcaagggcccttgggcggcgtcattgccctctccacctacgcgccgactttcggcgacgagctggaattgtctgccagccagcaacgcattcccgccctgtgcctgcatggccagtacgacgacgtggtgcagaacgccatgggccgcagcgcttttgagcatttgaagagccgtggtgtcaccgtgacatggcaggaatacccaatgggccacgaagtgttacctcaagagatacacgacatcggtgcctggctggccgctcgcctgggctga

SEQ ID NO: 2 - DNA, 654 bp, synthetic construct
  source 1..654: mol_type = other DNA
atgaccgaaccgctgattctgcaaccggcaaaaccggcagatgcatgtgttatttggctgcatggtctgggtgcagatcgttatgattttatgccggtggccgaagcactgcaagaaagcctgctgaccacccgttttgttctgccgcaggcaccgacccgtccggttacaattaatggtggttatgaaatgccgagctggtatgatattaaagcacatagtccggcacgtagcattagcctggaagaactggaagttagcgcaaaaatggttaccgatctgattgaagcacagaaacgtaccggtattgatgcaagccgtatttttctggcaggttttagccagggtggtgcagttgtttttcataccgcctttattaactggcagggtccgctgggtggtgttattgcactgagcacctatgcaccgacctttggtgatgaactggaactgagcgccagccagcagcgtattccggcactgtgtctgcatggccagtatgatgatgttgttcagaatgcaatgggtcgtagcgcatttgaacatctgaaaagccgtggtgttaccgttacctggcaagaatatccgatgggtcatgaagtgctgccgcaagaaattcatgatattggtgcatggctggcagcacgtctgggc